(19) United States
(12) Patent Application Publication
(10) Pub. No.: US 2024/0398833 A1
(43) Pub. Date: Dec. 5, 2024

(54) PHARMACEUTICAL COMPOSITION CONTAINING 27-HYDROXYCHOLESTEROL FOR PREVENTING OR TREATING MYELOID LEUKEMIA

(71) Applicant: PUSAN NATIONAL UNIVERSITY INDUSTRY-UNIVERSITY COOPERATION FOUNDATION, Geumjeong-gu Busan (KR)

(72) Inventors: Dongjun LEE, Gyeongsangnam-do (KR); Soo Yeon WOO, Gyeongsangnam-do (KR)

(21) Appl. No.: 18/699,698

(22) PCT Filed: Sep. 19, 2022
(86) PCT No.: PCT/KR2022/013934

§ 371 Date: Apr. 9, 2024

(30) Foreign Application Priority Data
Oct. 19, 2021 (KR) 10-2021-0139360
Jan. 21, 2022 (KR) 10-2022-0009248

(51) Int. Cl. A61K 31/575; A23K 20/168; A61K 8/63; A61P 35/02; A61Q 19/00

(52) U.S. Cl. CPC A61K 31/575; A23K 20/168; A61K 8/63; A61P 35/02; A61Q 19/00

(57) ABSTRACT

The present invention relates to a pharmaceutical composition, containing 27-hydroxycholesterol, for preventing or treating myeloid leukemia, and more specifically, to a pharmaceutical composition which suppresses the proliferation of hematopoietic stem cells by increasing the active oxygen of the hematopoietic stem cells and activating a cascade of endoplasmic reticulum stress, and promotes the apoptosis of blood cancer cells by activating the IRE1a, eIF2a, and CHOP signaling pathways which are important for the apoptosis of blood cancer cells.

### TECHNICAL FIELD

The present invention relates to a pharmaceutical composition containing 27-hydroxycholesterol for preventing or treating myeloid leukemia.

### BACKGROUND ART

Leukemia is a general term for diseases in which white blood cells proliferate in a neoplastic manner. Types of leukemia are classified into myeloid leukemia and lymphocytic leukemia depending on white blood cells from which leukemia originates, and into acute leukemia and chronic leukemia depending on a rate of progression. The clinical features of leukemia vary depending on a type of disease and the nature of invaded cells. The lymphocytic leukemia is caused by lymphoid blood cells, and the myeloid leukemia is caused by myeloid blood cells, and the chronic myeloid leukemia is caused by mutation of cells in the mature stage, and the acute myeloid leukemia is caused by a disorder of myeloid progenitor cells that begin differentiation during a relatively early stage of hematopoiesis.

Among these, the acute myeloid leukemia (AML) is a blood cancer in which abnormal leukemia cells that interfere with the production of normal white blood cells are produced and accumulated in the red bone marrow, and mainly occurs in adults and elderly people, and is known to account for about 70% of the overall acute leukemia. The symptoms of AML occur when the normal bone marrow is filled with leukemia cells and the number of blood cells (red blood cells, platelets, and normal white blood cells) rapidly decreases. Main symptoms include fatigue, rapid breathing, easy bruising or bleeding, frequent infections, and the like. Although various presumed causes of AML have been identified, the exact cause of AML has not been identified. The AML currently uses the classification of the World Health Organization (WHO), which includes more clinically important information such as cytogenetic abnormalities, and is subclassified into 1) genetically abnormal acute myeloid leukemia, 2) acute myeloid leukemia associated with myelodysplasia, 3) treatment-related acute myeloid leukemia, 4) unclassified acute myeloid leukemia, and 5) myeloid sarcoma. When diagnosed with acute myeloid leukemia, anticancer treatment is received, and first, complete remission using anticancer agents is induced. The complete remission is a stage in which cancer cells completely disappear, and partial remission means that more than 50% of cancer cells have disappeared. When an anticancer agent is administered to a patient, leukemia cells in the bone marrow die, and at the same time, normal cells are temporarily damaged, resulting in anemia and a decrease in platelets, and a decrease in white blood cells causes a sharp decline in immune function to increase the risk of infection from outside. When anticancer treatment with the anticancer agent is completed, leukemia cells disappear from the bone marrow, normal cells are regenerated in the bone marrow, and blood appearance becomes normal, leading to complete remission.

Efforts to treat the leukemia are continuing around the world, but serious life-threatening infection or bleeding complications caused by reduction in leukocytes and platelets due to damage to normal hematopoietic stem cells that are accompanied by anticancer treatment, and leukemia recurrence due to drug resistance are still a major barrier to leukemia treatment.

Therefore, the present inventors confirmed that 27-hydroxycholesterol suppressed the proliferation of hematopoietic stem cells by increasing the active oxygen of the hematopoietic stem cells and activating a cascade of endoplasmic reticulum stress, and promoted the apoptosis of blood cancer cells by activating IRE1a, eIF2a, and CHOP signaling pathways which were important for the apoptosis of blood cancer cells, and then completed the present invention by focusing on its applicability in the treatment of myeloid leukemia.

### DISCLOSURE

### Technical Problem

An object of the present invention is to provide a pharmaceutical composition for preventing or treating myeloid leukemia (ML) including 27-hydroxycholesterol or a pharmaceutically acceptable salt thereof as an active ingredient.

Another object of the present invention is to provide a method for preventing or treating myeloid leukemia (ML) including administering 27-hydroxycholerol to a subject in need thereof.

The objects to be solved by the present invention are not limited to the aforementioned object(s), and other object(s), which are not mentioned above, will be apparent to those skilled in the art from the following description.

### Technical Solution

An aspect of the present invention provides a pharmaceutical composition for preventing or treating myeloid leukemia (ML) including 27-hydroxycholesterol or a pharmaceutically acceptable salt thereof as an active ingredient.

The 27-hydroxycholesterol may suppress the proliferation of hematopoietic stem cells.

The proliferation of the hematopoietic stem cells may be suppressed by endoplasmic reticulum (ER) stress caused by the production of reactive oxygen species (ROS).

The 27-hydroxycholesterol may induce apoptosis of blood cancer cells.

The apoptosis of the blood cancer cells may be caused by endoplasmic reticulum (ER) stress induced by increased expression of one or more genes selected from the group consisting of inositolrequiring kinase 1α (IRE1α), eukaryotic translation initiation factor 2α (Elf2α), and C/EBP homologous protein (CHOP).

Another aspect of the present invention provides a health functional food composition for preventing or improving myeloid leukemia (ML) including 27-hydroxycholesterol or a pharmaceutically acceptable salt thereof as an active ingredient.

Yet another aspect of the present invention provides a feed composition for preventing or improving myeloid leukemia (ML) including 27-hydroxycholesterol or a pharmaceutically acceptable salt thereof as an active ingredient.

Yet another aspect of the present invention provides a cosmetic composition for preventing or improving myeloid leukemia (ML) including 27-hydroxycholesterol or a pharmaceutically acceptable salt thereof as an active ingredient.

Yet another aspect of the present invention provides an anticancer adjuvant for treating myeloid leukemia (ML) including 27-hydroxycholesterol or a pharmaceutically acceptable salt thereof as an active ingredient.

Yet another aspect of the present invention provides a method for preventing or treating myeloid leukemia (ML) including administering 27-hydroxycholerol to a subject in need thereof.

### Advantageous Effects

According to the present invention, the 27-hydroxycholesterol may suppress the proliferation of hematopoietic stem cells by increasing the active oxygen of the hematopoietic stem cells and activating a cascade of endoplasmic reticulum stress, and promote the apoptosis of blood cancer cells by activating IRE1a, eIF2a, and CHOP signaling pathways which are important for the apoptosis of blood cancer cells, and thus can be widely used in various fields of medicine, food, cosmetics and feed to prevent, improve or treat myeloid leukemia.

It should be understood that the effects of the present invention are not limited to the effects described above, but include all effects that can be deduced from the detailed description of the present invention or configurations of the disclosure described in claims.

### DESCRIPTION OF DRAWINGS

FIG. 1 illustrates results of confirming effects of 27-hydroxycholesterol on decreasing hematopoietic stem and progenitor cells (HSPC): (A) Flow cytometry result showing the proportions of LKS cells, hematopoietic progenitor cells (HPC), and hematopoietic stem cells (HSC); (B) Graph showing changes in proportions of Lin⁻Sca1⁺cKit⁺ cells (LKS), hematopoietic progenitor cells (Lin⁻Sca1⁺cKit⁺CD48⁺), and hematopoietic stem cells (Lin⁻Sca1⁺cKit⁺CD150⁺CD48⁻) according to treatment of 27-hydroxycholesterol in bone marrow cells (BM cells); (C) Flow cytometry result showing the proportions of common myeloid progenitor cells (CMP), granulocytic macrophage progenitor cells (GMP), megakaryocytic progenitor cells (MEP), and common lymphocytic progenitor cells (CLP); (D) Graph showing changes in proportions of common myeloid progenitor cells (Lin⁻Sca1⁻cKit⁺CD34⁺CD16/32⁻), granulocytic macrophage progenitor cells (Lin⁻Sca1⁻cKit⁺CD34⁺CD16/32⁺), megakaryocytic erythroid progenitor cells (Lin⁻Sca1⁻cKit⁺CD34⁻CD16/32⁻), and common lymphocytic progenitor cells (Lin⁻ Sca1^{low}cKit^{low} CD127⁺) according to treatment of 27-hydroxycholesterol in BM cells; (E) Flow cytometry result showing the proportions of monocytes, neutrophils, B cells, and T cells; and (F) Graph showing changes in proportions of monocytes (CD11b⁺), neutrophils (CD11b⁺Gr1⁺), B cells (B220⁺), and T cells (CD3⁺) according to treatment of 27-hydroxycholesterol in BM cells.

FIG. 2 illustrates results of confirming effects of 27-hydroxycholesterol on increased apoptosis and reactive oxygen species (ROS) in hematopoietic stem and progenitor cells (HSPC): (A) Flow cytometry result showing the apoptosis proportions of LK cells, LKS cells, hematopoietic progenitor cells (HPC), and hematopoietic stem cells (HSC); (B) Graph showing increased apoptosis proportions in LK cells, LKS cells, hematopoietic progenitor cells (HPC), and hematopoietic stem cells (HSC) treated with 27-hydroxycholesterol compared to control cells; (C) Flow cytometry result showing the ROS proportions of LK cells, LKS cells, hematopoietic progenitor cells (HPC), and hematopoietic stem cells (HSC); (D) Graph showing an increased ROS proportions in LK cells, LKS cells, hematopoietic progenitor cells (HPC), and hematopoietic stem cells (HSC) treated with 27-hydroxycholesterol compared to control cells; (E) Graph showing an effect of N-acetylcysteine (NAC) on reducing reactive oxygen species (ROS) in LKS cells, hematopoietic progenitor cells (HPC), and hematopoietic stem cells (HSC) increased by 27-hydroxycholesterol; and (F) Graph showing an effect of N-acetylcysteine (NAC) on suppressing the decreased proportions of LKS cells, hematopoietic progenitor cells (HPC), and hematopoietic stem cells (HSC) by 27-hydroxycholesterol.

FIG. 3 illustrates results of confirming an effect of 27-hydroxycholesterol on leukemia cell growth in vitro: (A) Graph showing changes in cell count of myeloid leukemia cells (HL60, KG1a, and K562 cells) according to treatment of 27-hydroxycholesterol [Con (negative control): HEK293T cells]; (B) Flow cytometry result showing apoptosis proportions of myeloid leukemia cells (HL60, KG1a, and K562 cells) according to treatment of 27-hydroxycholesterol; and (C) Graph showing increased apoptosis proportions in myeloid leukemia cells (HL60, KG1a, and K562 cells) treated with 27-hydroxycholesterol compared to control cells.

FIG. 4 illustrates results of confirming an effect of 27-hydroxycholesterol on leukemia cell growth in vitro: (A) Flow cytometry result showing ROS proportions of myeloid leukemia cells (HL60, KG1a, and K562 cells) treated with 27-hydroxycholesterol; (B) Graph showing increased ROS proportions in myeloid leukemia cells (HL60, KG1a, and K562 cells) treated with 27-hydroxycholesterol compared to control cells; (C) Flow cytometry result showing differentiation (CD11b⁺) proportions of myeloid leukemia cells (HL60, KG1a, and K562 cells) according to treatment of 27-hydroxycholesterol; and (D) Graph showing differentiation (CD11b⁺) proportions in myeloid leukemia cells (HL60, KG1a, and K562 cells) treated with 27-hydroxycholesterol compared to control cells.

FIG. 5 illustrates results of confirming an effect of 27-hydroxycholesterol on leukemia cell growth in vitro: (A) Western blot image showing an increased ER stress response pathway of 27-hydroxycholesterol in THP1 cells; and (B) Schematic diagram showing an apoptosis inducing mechanism of 27-hydroxycholesterol.

FIG. 6 illustrates results of confirming a gene expression level according to treatment of 27-hydroxycholesterol: (A) Graph showing an expression level of an Era gene related to cancer cell growth in LKS cells and hematopoietic progenitor cells (HPC) according to treatment of 27-hydroxycholesterol; (B) Graph showing an expression level of a Bax gene related to apoptosis in LKS cells and hematopoietic progenitor cells (HPC) according to treatment of 27-hydroxycholesterol; and (C) Graph showing expression levels of Chop, Ire1α, and Xbp1s genes related to ER stress in LKS cells and hematopoietic progenitor cells (HPC) according to treatment of 27-hydroxycholesterol.

FIG. 7 shows graphs of confirming changes in proportions of (A) Lin-Sca1⁺cKit⁺ cells (LKS), hematopoietic stem cells (HSC, Lin⁻Sca1⁺cKit⁺CD150⁺CD48-), and hematopoietic progenitor cells (HPC, Lin⁻Sca1⁺cKit⁺CD48⁺), (B) granulocytic macrophage progenitor cells (GMP, Lin⁻Sca1⁻cKit⁺CD34⁺CD16/32⁺), common myeloid progenitor cells (CMP, Lin⁻Sca1⁻cKit⁺CD34⁺CD16/32⁻), common lymphocytic progenitor cells (CLP, Lin⁻ Sca1^{low} cKit^{low} CD127⁺), and megakaryocytic erythroid progenitor cells (MEP, Lin⁻Sca1⁻cKit⁺CD34⁻CD16/32⁻), and (C) monocytes (CD11b⁺), neutrophil (CD11b⁺Gr1⁺), B cells (B220⁺), and T cells (CD3⁺) according to inoculation of 27-hydroxycholesterol into the mouse cavity, as a result of in vivo experiments according to injection of 27-hydroxycholesterol into mice.

FIG. 8 is a graph showing a result of comparing leukemia cell growth between 27-hydroxycholesterol and 7α-hydroxycholesterol in (A) HL60 cells, (B) K562 cells, and (C) HEK293T cells.

FIG. 9 illustrates results of confirming an effect of 27-hydroxycholesterol on regeneration and proliferation of hematopoietic stem cells ex vivo: (A) Experimental process; (B) Result graph of analyzing donor mouse CD45.1 cells in blood; (C) Graph of results of measuring the proportions of monocytes (Myelo; CD11b⁺), B cells (B220⁺), and T cells (CD3⁺) of a donor mouse in the blood of a recipient mouse after transplantation; (D) Result graph of analyzing donor mouse CD45.1 cells in bone marrow cells; and (E) Result graph of measuring the proportions of LK cells, LKS cells, hematopoietic progenitor cells (HPC), and hematopoietic stem cells (HSC) of a donor mouse in the bone marrow cells of a recipient mouse after transplantation.

FIG. 10 illustrates results of RNA-sequence analysis of an acute myeloid leukemia data sheet GSE116256: (A) Graph showing viability curves related to CYP7B1 expression based on an acute myeloid leukemia data sheet GSE116256; (B) Result graph of confirming the expression of CYP7B1 in BM cells of a patient with acute myeloid leukemia (AML) and a healthy donor (Healthy); (C) Graph showing hematopoietic cell composition analysis of a patient with acute myeloid leukemia (AML) and a healthy donor (Healthy); (D) Graph showing the expression levels of CYP7B1 in all malignant cells, hematopoietic stem cells (HSC), dendritic cells (cDC), and monocytes (mono) of a patient with acute myeloid leukemia; (E) Biological process of genes differentially correlated with CYP7B1 in normal and malignant hematopoietic stem cells (HSC); and (F) Biological process of genes differentially correlated with CYP7B1 in normal and malignant dendritic cells (cDC).

FIG. 11 illustrates results of confirming effects of 27-hydroxycholesterol on decreasing hematopoietic stem and progenitor cells (HSPC): (A) Graph showing effects over time in LK cells, LKS cells, hematopoietic progenitor cells (HPC), and hematopoietic stem cells (HSC); (B) Graph showing effects according to concentrations in LK cells, LKS cells, hematopoietic progenitor cells (HPC), hematopoietic stem cells (HSC), monocytes (CD11b⁺), neutrophils (CD11b⁺Gr1⁺), B cells (B220⁺), and T cells (CD3⁺); and (C) Graph showing the reduction effects on cKit⁺ cells and CD48⁺ cells.

FIG. 12 is a schematic diagram showing a signaling pathway of inducing the apoptosis of blood cancer cells.

### MODES OF THE INVENTION

Hereinafter, exemplary embodiments of the present invention will be described in more detail with reference to the accompanying drawings. The exemplary embodiments of the present invention can be modified in various forms, and it should not be construed that the scope of the present invention is limited to exemplary embodiments to be described below. The exemplary embodiments will be provided for more completely explaining the present invention to those skilled in the art. The exemplary embodiments will be provided for more completely explaining the present invention to those skilled in the art.

The present invention provides a pharmaceutical composition for preventing or treating myeloid leukemia (ML) including 27-hydroxycholesterol or a pharmaceutically acceptable salt thereof as an active ingredient.

The 27-hydroxycholesterol may be a compound represented by the following Chemical Formula 1.

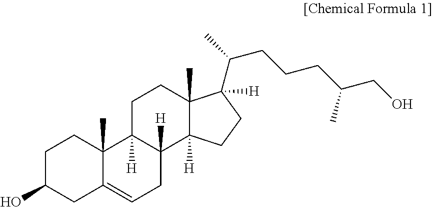

The 27-hydroxycholesterol is an endogenous oxysterol with diverse biological functions, including activity as a selective estrogen receptor modulator (SERM) and an agonist of liver X receptor (LXR), and known as a metabolite of cholesterol produced by an enzyme CYP27A1. In the present invention, it was confirmed that the 27-hydroxycholesterol was effective in preventing, improving, or treating myeloid leukemia, and myeloid leukemia containing 27-hydroxycholesterol as an active ingredient, which is intended to be applied as a pharmaceutical composition for preventing, improving, or treating myeloid leukemia including 27-hydroxycholesterol as an active ingredient.

According to an exemplary embodiment of the present invention, the 27-hydroxycholesterol may suppress the proliferation of hematopoietic stem cells. Specifically, the proliferation of the hematopoietic stem cells may be suppressed by endoplasmic reticulum (ER) stress caused by the production of reactive oxygen species (ROS).

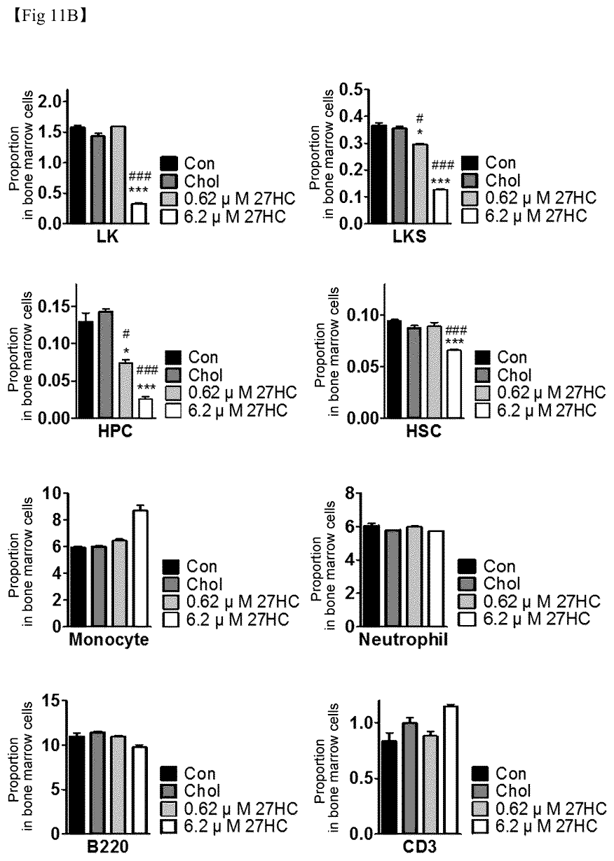

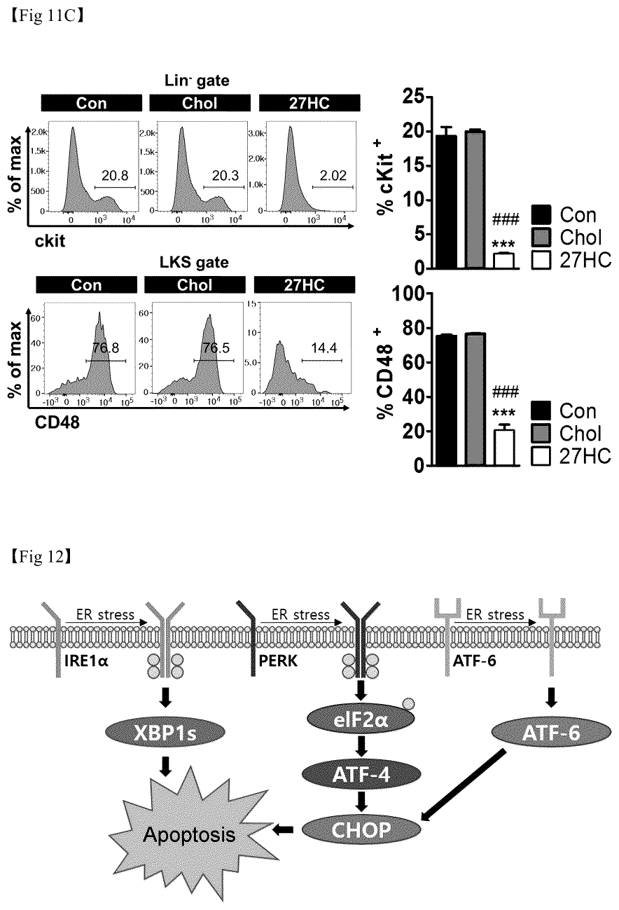

According to an exemplary embodiment of the present invention, the 27-hydroxycholesterol may induce the apoptosis of blood cancer cells, suppress the stemness of blood cancer stem cells, or suppress the metastasis of the blood cancer cells or cancer stem cells. Specifically, the apoptosis of the blood cancer cells may be caused by endoplasmic reticulum (ER) stress induced by increased expression of one or more genes selected from the group consisting of inositolrequiring kinase 1α (IRE1α), eukaryotic translation initiation factor 2α (Elf2α), and C/EBP homologous protein (CHOP). More specifically, the apoptosis may be caused by activation of the IRE1a, eIF2a, and CHOP signaling pathways. This will be described in detail with reference to FIG. 12.

Referring to FIG. 12, there are two types of ER of rough ER attached with ribosomes and smooth ER without ribosomes. In the rough ER, RNA is converted into a protein through various processes. However, when immature proteins that exceed the ability to be processed by ER are introduced into the ER, dysfunction of the ER occurs, which is called ER stress. When the ER stress occurs, cells have defense mechanisms to survive. When the ER stress occurs, the membrane proteins IRE1α, PERK, and ATF-6 present in the ER membrane activate the apoptosis pathway to remove damaged cells. The activated IRE1α may induce the apoptosis by activating XBP1, and the activation of PERK and ATF-6 may induce the apoptosis by activating elF2α, ATF-4, and ATF-6 to activate CHOP, respectively.

According to an exemplary embodiment of the present invention, the concentration of the 27-hydroxycholesterol or the pharmaceutically acceptable salt thereof may be 1 to 20 μM, 1 to 10 μM, 3 to 9 μM, or 5 to 7 μM. Within the range, there is an effect of more improving the ability to induce the apoptosis by 27-hydroxycholesterol, generate the reactive oxygen species, and suppress the proliferation of hematopoietic stem cells.

In addition, the present invention provides a method for inducing apoptosis including contacting myeloid leukemia cells with 27-hydroxycholesterol or a pharmaceutically acceptable salt thereof in vitro or ex vivo.

In addition, the present invention provides a method for increasing reactive oxygen species including contacting myeloid leukemia cells with 27-hydroxycholesterol or a pharmaceutically acceptable salt thereof in vitro or ex vivo.

In addition, the present invention provides a method for suppressing the proliferation of hematopoietic stem cells including contacting myeloid leukemia cells with 27-hydroxycholesterol or a pharmaceutically acceptable salt thereof in vitro or ex vivo.

In the method for inducing the apoptosis, the method for increasing the reactive oxygen species, and the method for suppressing the proliferation of hematopoietic stem cells, the concentration of 27-hydroxycholesterol or the pharmaceutically acceptable salt thereof may be 1 to 20 μM, 1 to 10 μM, 3 to 9 μM, or 5 to 7 μM. Within the range, there is an effect of more improving the ability to induce the apoptosis by 27-hydroxycholesterol, generate the reactive oxygen species, and suppress the proliferation of hematopoietic stem cells.

As used herein, the term “prevention” refers to all actions that delay the progression of myeloid leukemia by suppressing the proliferation of hematopoietic stem cells or inducing the apoptosis of blood cancer cells by administering the composition of the present invention.

As used herein, the term “treatment” refers to all actions that improve or beneficially change myeloid leukemia by suppressing the proliferation of hematopoietic stem cells or inducing the apoptosis of blood cancer cells by administering the composition of the present invention, and refers to attempts to obtain useful or desirable results, including clinical results. Although detectable or not, the useful or desirable clinical results may include alleviation or improvement of one or more symptoms or conditions, reduction of the range of diseases, stabilization of the disease condition, inhibition of the occurrence of the disease, inhibition of the spread of the disease, delay or slowing of the progression of the disease, delay or slowing of the onset of the disease, improvement or alleviation of the disease condition, and decay (partial or total), but are not necessarily limited thereto. In addition, the “treatment” may mean prolonging the patient's survival beyond what is expected in the absence of treatment. In addition, the “treatment” may refer to inhibiting the progression of the disease, and temporarily slowing the progression of the disease, and more preferably, permanently stopping the progression of the disease. In the present invention, the “treatment” may preferably mean improving patient survival by improving blood cancer, especially myeloid leukemia.

The pharmaceutical composition of the present invention may be formulated and used in the form of oral formulations such as powders, granules, tablets, capsules, suspensions, emulsions, syrups, aerosols, etc., external preparations, suppositories, and sterile injectable solutions according to conventional methods, respectively. The carrier, the excipient, and the diluent that may be included in the pharmaceutical composition may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil. When the pharmaceutical composition is formulated, the formulation may be prepared by using diluents or excipients, such as a filler, an extender, a binder, a wetting agent, a disintegrating agent, and a surfactant, which are generally used. Solid formulations for oral administration include tablets, pills, powders, granules, capsules, and the like, and these solid formulations may be prepared by mixing at least one excipient, for example, starch, calcium carbonate, sucrose or lactose, gelatin, and the like with the compound of the present invention. Further, lubricants such as magnesium stearate and talc are used in addition to simple excipients. Liquid formulations for oral administration may correspond to suspensions, oral liquids, emulsions, syrups, and the like, and may include various excipients, for example, a wetting agent, a sweetener, an aromatic agent, a preservative, and the like, in addition to water and liquid paraffin which are commonly used as simple diluents. Formulations for parenteral administration include sterilized aqueous solutions, non-aqueous solvents, suspensions, emulsions, lyophilized agents, and suppositories. As the non-aqueous solution and the suspension, propylene glycol, polyethylene glycol, vegetable oil such as olive oil, injectable ester such as ethyl oleate, and the like may be used. As a base of the suppository, witepsol, macrogol, tween 61, cacao butter, laurinum, glycerogelatin, and the like may be used.

The dose of the pharmaceutical composition of the present invention will vary depending on the age, sex, and body weight of a subject to be treated, a specific disease or pathological condition to be treated, the severity of the disease or pathological condition, a route of administration, and the judgment of a prescriber. The dose based on these factors is determined within a level of those skilled in the art, and in general, the dose is in the range of 0.01 mg/kg/day to about 2000 mg/kg/day. A more preferable dose is 0.1 mg/kg/day to 1000 mg/kg/day. The dose may be administered once a day or several times a day. The dose does not limit the scope of the present invention in any aspect.

The pharmaceutical composition of the present invention may be administered to mammals such as rats, livestock, and human in various routes. All methods of administration may be expected and for example, the pharmaceutical composition may be administered by oral, rectal or intravenous, intramuscular, subcutaneous, intrauterine dural or cerebrovascular injection.

In the present invention, in addition to the active ingredient, the pharmaceutical composition for preventing or treating myeloid leukemia may further include any compound or natural extract that has already been proven in safety and known to have anticancer activity in order to increase and reinforce the anticancer effect.

Further, the present invention provides a food composition or health functional food composition for preventing or improving myeloid leukemia (ML) including 27-hydroxycholesterol or a pharmaceutically acceptable salt thereof as an active ingredient.

The food or health functional food composition may further include an additive selected from the group consisting of flavoring agents, colorants, fillers, stabilizers, natural carbohydrates, nutrients, vitamins, thickeners, pH adjusters, preservatives, and mixtures thereof.

The food composition of the present invention includes all forms, such as functional foods, nutritional supplements, health foods, and food additives. The type of food composition may be prepared in various forms according to conventional methods known in the art.

For example, as the health food, the composition itself may be prepared in the form of tea, juice, and drinks to be drunk, or taken in by granulation, encapsulation and powder. In addition, the functional food may be prepared by adding the extract to beverages (including alcoholic beverages), fruits and processed foods thereof (e.g., canned fruit, bottled food, jam, marmalade, etc.), fish, meat and processed foods thereof (e.g., ham, sausage, corned beef, etc.), bread and noodles (e.g., udon, buckwheat noodles, ramen, spaghetti, macaroni, etc.), fruit juice, various drinks, cookies, sweets, dairy products (e.g., butter, cheese, etc.), edible vegetable oil, margarine, vegetable protein, retort food, frozen food, various seasonings (e.g., soybean paste, soy sauce, sauce, etc.), etc. In order to use the composition of the present invention in the form of food additives, the composition may be prepared and used in the form of powders or concentrates.

The preferred content of 27-hydroxycholesterol in the food composition of the present invention may be 0.001 to 50%, preferably 0.01 to 30%, based on the total weight of the food composition.

In an exemplary embodiment of the present invention, the health functional food composition of the present invention may be prepared in general formulations, such as tablets, pills, granules, powders, liquids, hard capsules, soft capsules, etc., and may be prepared in any form such as porridge, bread, beverages, bars, chocolate, cookies, tea, drinks, vitamin complex, meat, sausage, candy, noodles, jelly, etc.

In order to prepare various formulations or forms as described above, food-acceptable carriers or additives such as the above-mentioned excipients may be used, and may be used with any carrier or additive known in the art to be usable in the art in the preparation of the formulations or forms to be prepared.

Further, the present invention provides a feed composition for preventing or improving myeloid leukemia (ML) including 27-hydroxycholesterol or a pharmaceutically acceptable salt thereof as an active ingredient.

When the 27-hydroxycholesterol of the present invention is provided in the form of a feed composition, the feed composition may additionally include known feed supplements, food additives, or feed additives, and may be prepared in the form such as fermented feed, compounded feed, pellets, silage, etc.

Further, the present invention provides a cosmetic composition for preventing or improving myeloid leukemia (ML) including 27-hydroxycholesterol or a pharmaceutically acceptable salt thereof as an active ingredient.

When the 27-hydroxycholesterol of the present invention is provided in a cosmetic composition, the cosmetic composition may include, without limitation, other commonly accepted ingredients in addition to the active ingredient, and may include conventional adjuvants such as antioxidants, stabilizers, solubilizers, vitamins, pigments and flavors, and carriers. The cosmetic composition may be interpreted as including various materials for skin health without limitation.

Further, the present invention provides an adjuvant for treating myeloid leukemia (ML) including 27-hydroxycholesterol or a pharmaceutically acceptable salt thereof as an active ingredient.

As used herein, the term “adjuvant for treating myeloid leukemia” refers to a composition that synergistically increases the effect of anticancer treatment by preventing side effects caused by the anticancer agent when applied in parallel with treatment with the anticancer agent. Therefore, the therapeutic adjuvant is an anticancer adjuvant and is able to be administered together with the anticancer agent, simultaneously or sequentially.

The type of anticancer agent that may be used with the adjuvant for treating myeloid leukemia of the present invention is not particularly limited. The anticancer agent may be selected under general principles considered when selecting anticancer agents, such as a type of cancer cell, an absorption rate of the anticancer agent (treatment period and route of anticancer agent administration), a location of the tumor, and a size of the tumor.

Further, the present invention provides a method for preventing or treating myeloid leukemia (ML) including administering 27-hydroxycholerol to a subject in need thereof.

As used herein, the term “subject” refers to a subject in need of hemostasis, and more particularly, may mean mammals such as humans or non-human primates, mice, dogs, cats, horses and cattle, but is not limited thereto.

The above description just illustrates the technical spirit of the present invention and various changes and modifications can be made by those skilled in the art to which the present invention pertains without departing from an essential characteristic of the present invention. Accordingly, the various exemplary embodiments disclosed in the present invention are not intended to limit the technical spirit but describe the present invention and the technical spirit of the present invention is not limited by the following exemplary embodiments. The protective scope of the present invention should be construed based on the following claims, and all the techniques in the equivalent scope thereof should be construed as falling within the scope of the present invention.

Hereinafter, the present invention will be described in more detail through Examples.

### Example 1. Confirmation of Effect of 27-Hydroxycholesterol on Decreased Hematopoietic Stem and Progenitor Cells (HSPC)

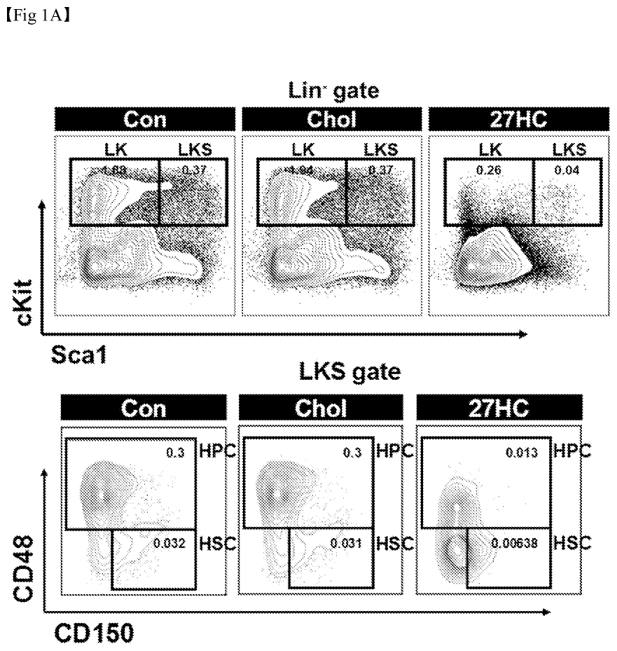

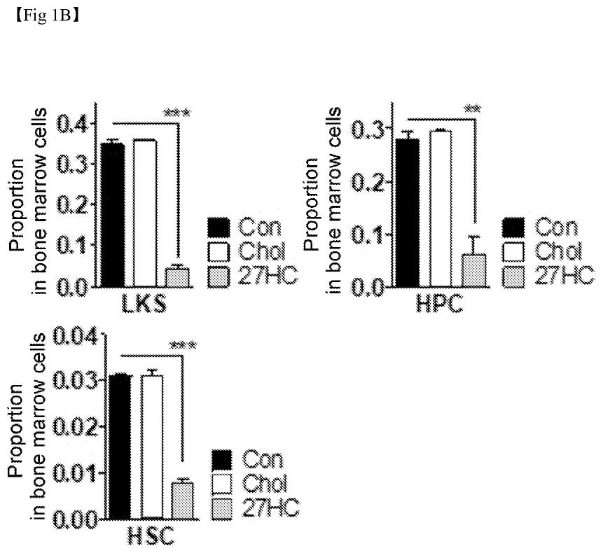

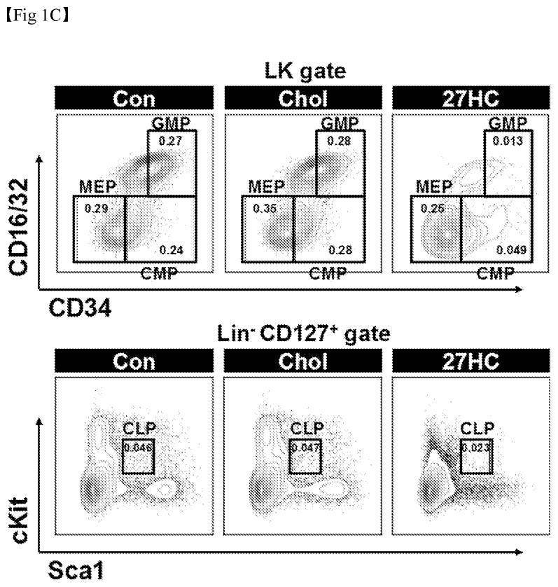

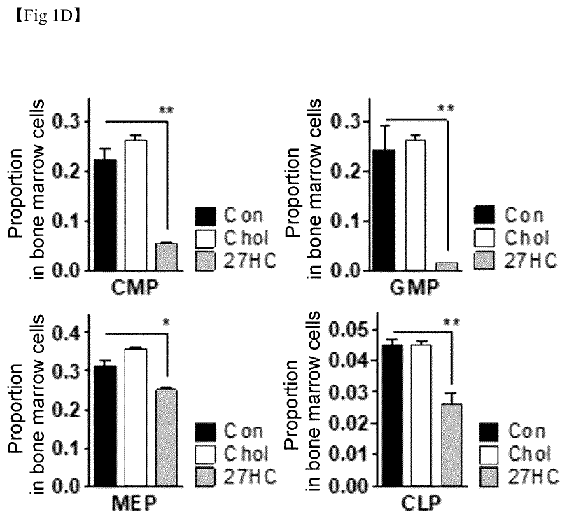

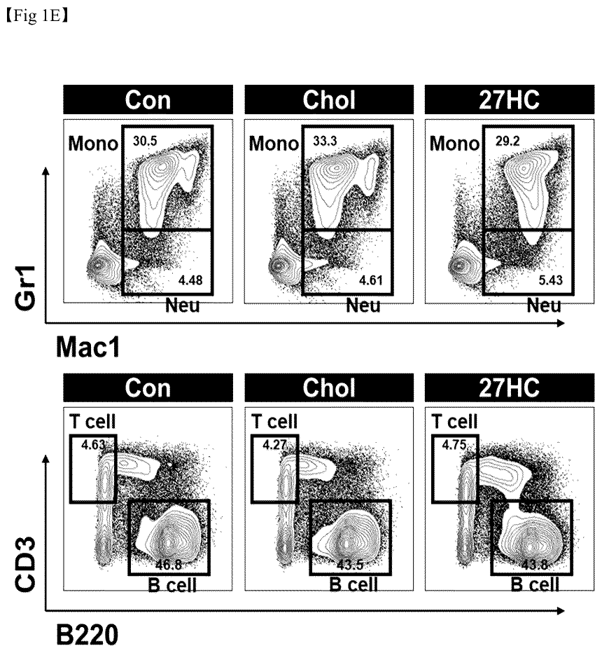

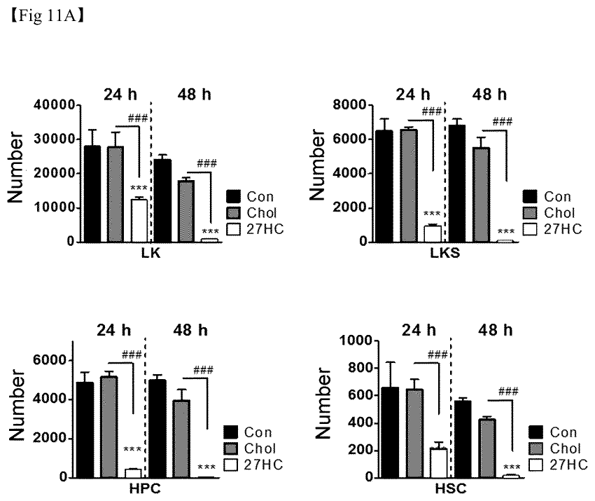

The bone marrow was extracted from the mouse tibia and femur using an FACS staining buffer (phosphate-buffered saline, 2% FBS, penicillin/streptomycin), and then bone marrow cells were extracted by lysing red blood cells using an ACK lysis buffer and treated with 0.62 μM or 6.2 μM 27-hydroxycholesterol for 24 or 48 hours. After treatment, antibodies were attached and the cells were analyzed using FACSCanto2 equipment. As a result, the effect of 27-hydroxycholesterol on decreased hematopoietic stem and progenitor cells (HSPC) was confirmed, and illustrated in FIGS. 1 and 11 [Con: untreated mouse bone marrow cells, Chol: mouse bone marrow cells treated with cholesterol, 27HC: mouse bone marrow cells treated with 27-hydroxycholesterol].

Referring to FIGS. 1A and 1, it was confirmed that as bone marrow cells (BM cells) were treated with 27-hydroxycholesterol, proportions of Lin⁻Sca1⁺cKit⁺ cells (LKS), hematopoietic progenitor cells (Lin⁻Sca1cKit⁺CD48⁺), and hematopoietic stem cells (Lin⁻Sca1cKit⁺CD150⁺CD48⁻) were decreased.

In addition, referring to FIGS. 1C and 1D, it was confirmed that as the BM cells were treated with 27-hydroxycholesterol, proportions of common myeloid progenitor cells (Lin⁻Sca1⁻cKit⁺CD34⁺CD16/32⁻), granulocytic macrophage progenitor cells (Lin⁻Sca1⁻cKit⁺CD34⁺CD16/32⁺), megakaryocytic erythroid progenitor cells (Lin⁻Sca1⁻cKit⁺CD34⁻CD16/32⁻), and common lymphocytic progenitor cells (Lin⁻ Sca1^{low}cKit^{low} CD127⁺) were decreased.

In addition, referring to FIGS. 1E and 1F, it was confirmed that as the BM cells were treated with 27-hydroxycholesterol, there was no change in proportions of monocytes (CD11b⁺), neutrophils (CD11b⁺Gr1⁺), B cells (B220⁺), and T cells (CD3⁺).

In addition, referring to FIG. 11A, it was confirmed that when the BM cells were treated with 27-hydroxycholesterol for 24 hours or 48 hours, the number of LK cells, LKS cells, hematopoietic progenitor cells (HPC), and hematopoietic stem cells (HSC) was decreased, and it was confirmed that there was a significant reduction effect over time.

In addition, referring to FIG. 11B, it was confirmed that when the BM cells were treated with 27-hydroxycholesterol for 24 hours at concentrations of 0.62 μM and 6.2 μM, respectively, the treatment of 0.62 μM of 27-hydroxycholesterol had no effect on all cells.

In addition, referring to FIG. 11C, it can be seen that 27-hydroxycholesterol reduces cKit⁺ cells and CD48⁺ cells.

Through this, it was confirmed that 27-hydroxycholesterol has a cytotoxic effect specifically on hematopoietic stem and progenitor cells.

### Example 2. Confirmation of Effect of 27-Hydroxycholesterol on Increased Apoptosis and Reactive Oxygen Species (ROS) in Hematopoietic Stem and Progenitor Cells (HSPC)

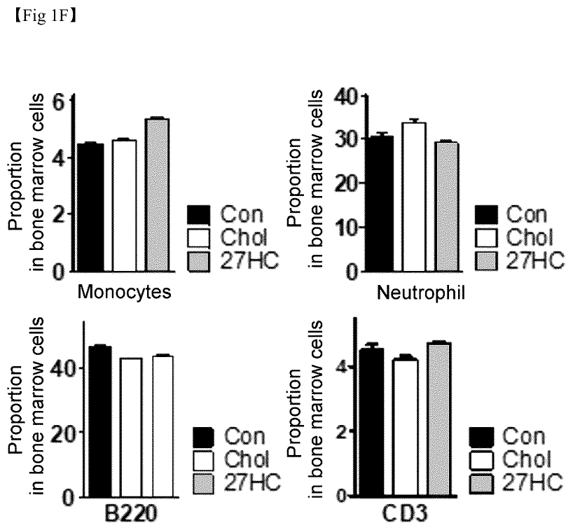

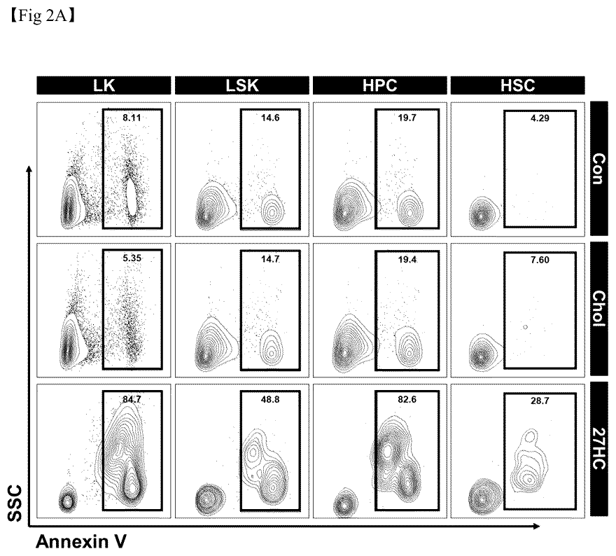

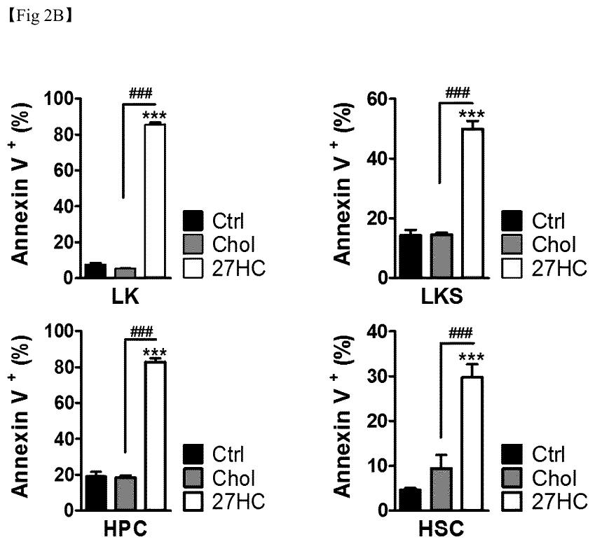

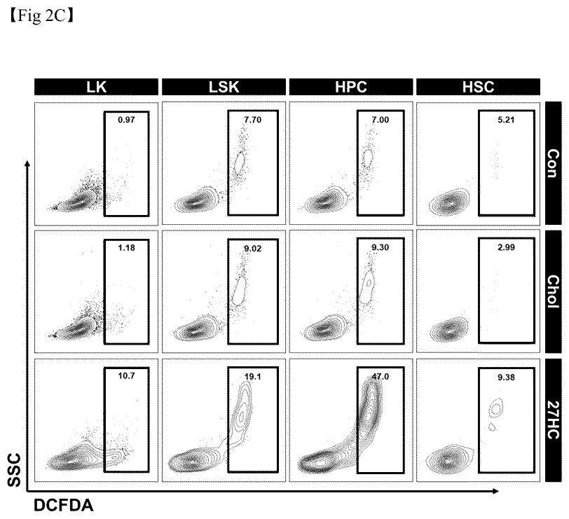

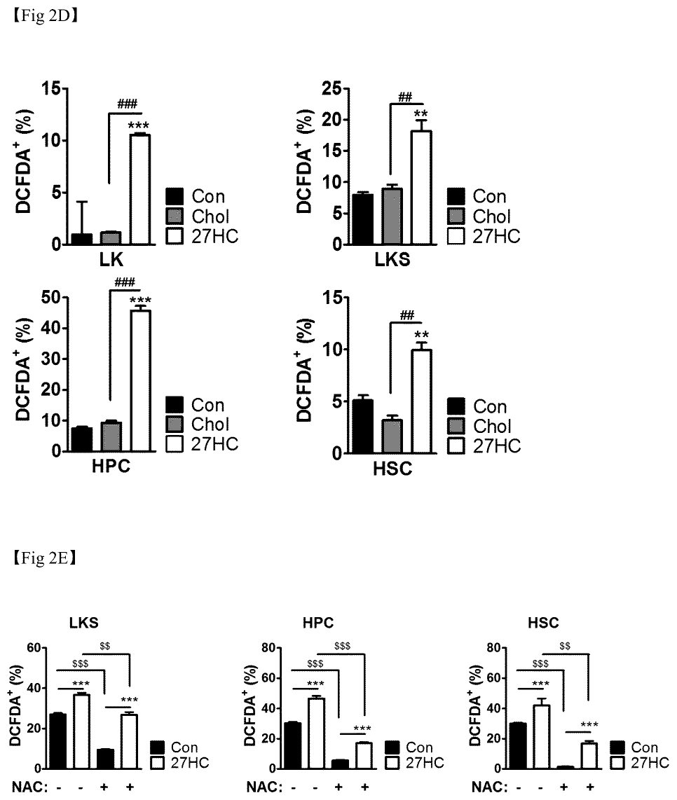

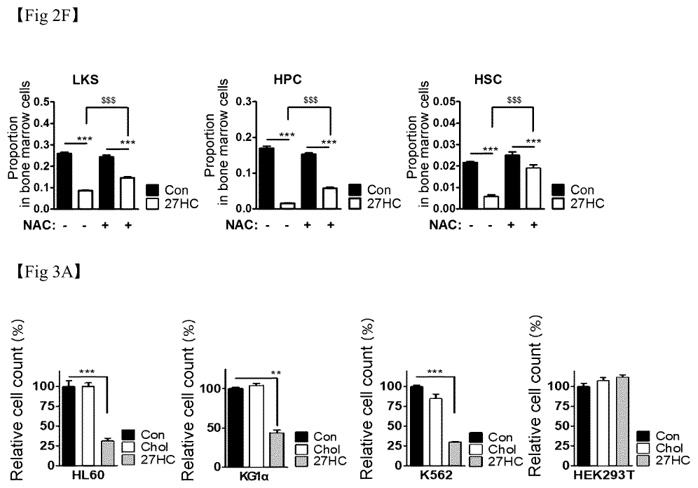

Bone marrow cells were extracted from the mouse tibia and femur and treated with 6.2 μM of 27-hydroxycholesterol for 48 hours. In order to confirm apoptosis, the cells treated with 27-hydroxycholesterol were collected, the cells were dissolved in a 1× Annexin binding buffer, added with Annexin V and 7AAD, and analyzed using FACScanto2 equipment, and in order to confirm reactive oxygen species, the cells were dissolved in an FACS staining buffer, added with DCFDA, stained at 37° C. for 30 minutes, and analyzed using FACScanto2 equipment. As a result, an effect of 27-hydroxycholesterol on increased apoptosis and reactive oxygen species (ROS) in hematopoietic stem and progenitor cells (HSPC) was confirmed and illustrated in FIG. 2 [Con or Ctrl: mouse bone marrow cells untreated, Chol: mouse bone marrow cells treated with cholesterol, 27HC: mouse bone marrow cells treated with 27-hydroxycholesterol].

Referring to FIGS. 2A and 2B, it was confirmed that compared to control cells, the apoptosis was increased in LKS cells, hematopoietic progenitor cells (HPC), and hematopoietic stem cells (HSC) treated with 27-hydroxycholesterol.

Referring to FIGS. 2C and 2D, it was confirmed that compared to control cells, the ROS was increased in LKS cells, hematopoietic progenitor cells (HPC), and hematopoietic stem cells (HSC) treated with 27-hydroxycholesterol.

In addition, referring to FIG. 2E, it was confirmed that compared to control cells, the ROS was increased in LKS cells, hematopoietic progenitor cells (HPC), and hematopoietic stem cells (HSC) treated with 27-hydroxycholesterol, and the ROS was increased by treating an antioxidant N-acetylcysteine (NAC).

In addition, referring to FIG. 2F, it was confirmed that compared to control cells, the proportions of LKS cells, hematopoietic progenitor cells (HPC), and hematopoietic stem cells (HSC) treated with 27-hydroxycholesterol were decreased, and the decreased proportions of the cells were suppressed by treating an antioxidant N-acetylcysteine (NAC).

Through this, it was confirmed that the 27-hydroxycholesterol increased the apoptosis and the ROS in hematopoietic stem and progenitor cells. Specifically, in hematopoietic stem and progenitor cells treated with cholesterol, the apoptosis and the ROS proportions were similar to those of untreated cells, whereas in cells treated with 27-hydroxycholesterol, the apoptosis and the ROS proportions were increased. As a result, it can be seen that the specific cytotoxicity of 27-hydroxycholesterol in hematopoietic stem and progenitor cells is caused by the increased apoptosis and ROS.

### Example 3. Confirmation of Effect of 27-Hydroxycholesterol on Leukemia Cell Growth In Vitro

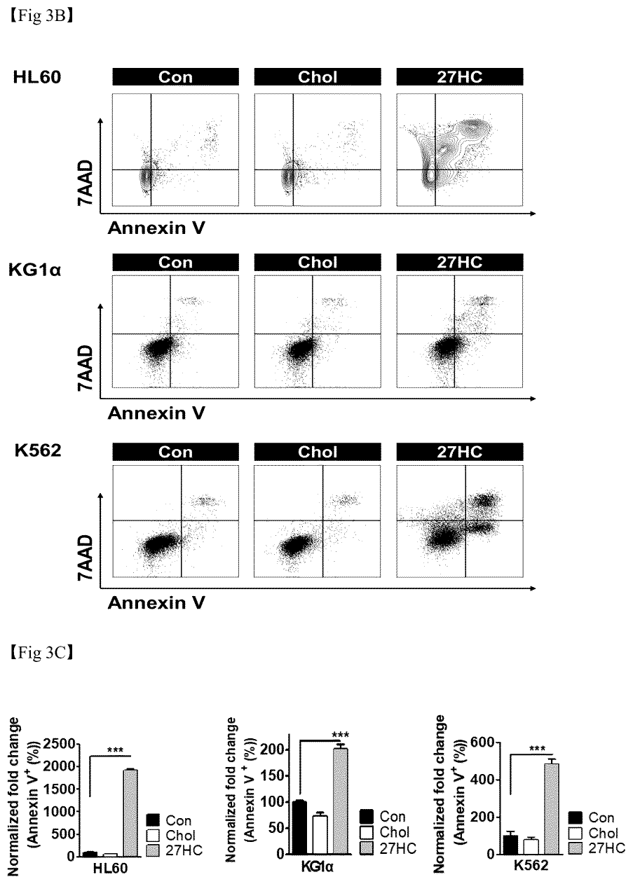

Acute myeloid leukemia cell lines HL60 and KG1 α, and chronic myeloid leukemia cell line, K562 cells were treated with 6.2 μM of 27-hydroxycholesterol for 48 hours and then strained with trypan blue, and then the cell count was confirmed. In addition, the cells were added with Annexin V and 7AAD, and the apoptosis was analyzed using FACScanto2 equipment. As a result, it was confirmed that compared to untreated cells (Con) and a cholesterol-treated leukemia cell line (Chol), 27-hydroxycholesterol increased the apoptosis in a leukemia cell line (27HC) to reduce the number of cells, which was illustrated in FIG. 3 [Con: untreated leukemia cell line, Chol: leukemia cell line treated with cholesterol, 27HC: leukemia cell line treated with 27-hydroxycholesterol].

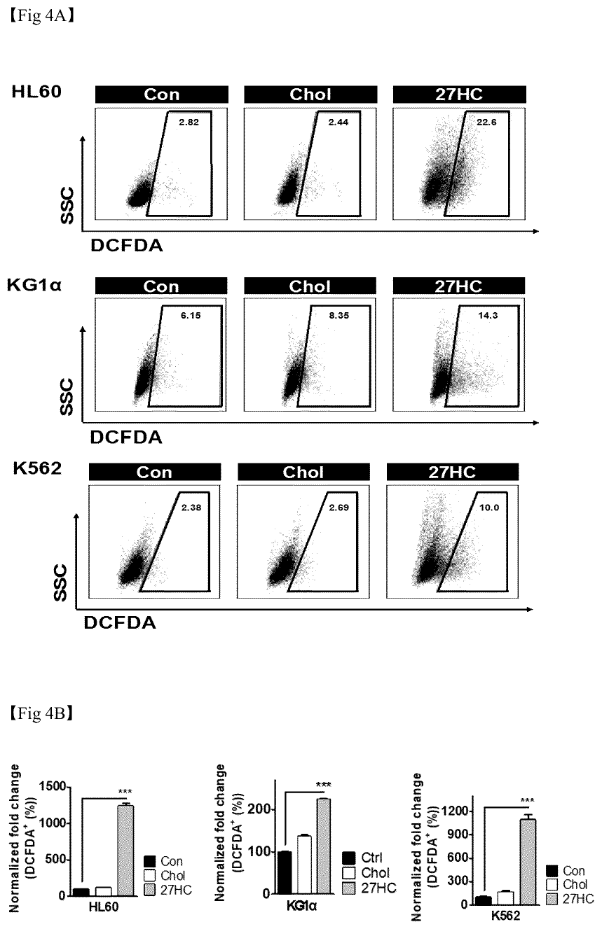

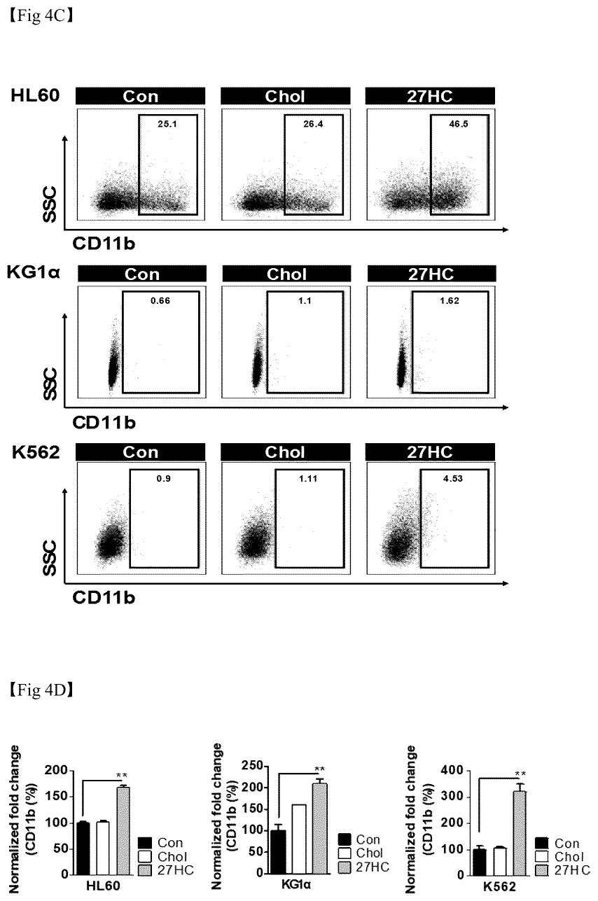

Next, the apoptosis and the ROS proportions were analyzed through DCFDA staining. In addition, to confirm the effect of 27-Hydroxycholesterol on the differentiation of leukemia cell lines, a differentiation marker, CD11b was stained and analyzed using FACScanto 2 equipment. As a result, it was confirmed that the ROS proportion was high in the leukemia cell line treated with 27-hydroxycholesterol, which was illustrated in FIG. 4 [Con: untreated leukemia cell line, Chol: leukemia cell line treated with cholesterol, 27HC: leukemia cell line treated with 27-hydroxycholesterol].

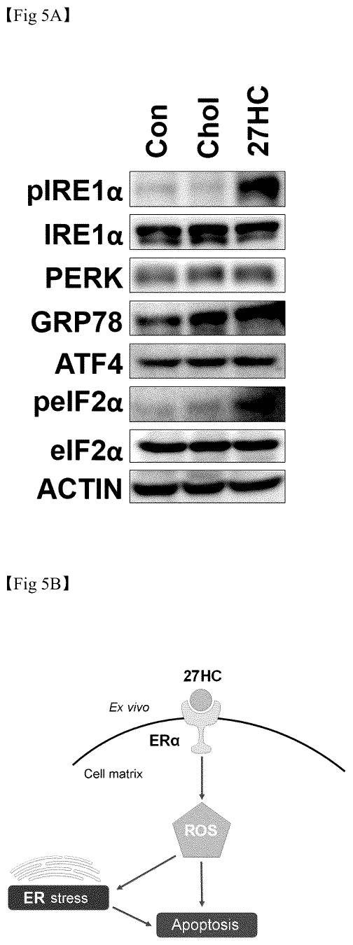

Next, in order to determine whether the apoptosis induced by 27-hydroxycholesterol was caused by ER stress, the amount of ER stress protein was confirmed in HL60 cells, an acute myeloid leukemia cell line. 27-hydroxycholesterol was treated for 48 hours, and then cells were lysed with a lysis buffer to extract proteins, the proteins were separated on a 10% SDS-PAGE gel and transferred to a membrane, and then added with anti-pIRE1α, IRE1α, PERK, ATF4, elF2α, or anti-ACTIN antibodies, stained at 4° C. for 16 hours, added with secondary antibodies, stained for 1 hour at room temperature, reacted with ECL, and measured using a chemiluminescence image analyzer. As a result, it was confirmed that 27-hydroxycholesterol increased ER stress, as shown in FIG. 5.

Referring to FIG. 3A, it was confirmed that as 27-hydroxycholesterol was treated, the number of myeloid leukemia cells (HL60, KG1a, and K562 cells) was significantly decreased compared to the number of HEK293T cells, which was a negative control.

In addition, referring to FIGS. 3B and 3C, it was confirmed that myeloid leukemia cells (HL60, KG1a, K562 cells) treated with 27-hydroxycholesterol showed increased apoptosis compared to control cells.

In addition, referring to FIGS. 4A and 4B, it was confirmed that myeloid leukemia cells (HL60, KG1a, K562 cells) treated with 27-hydroxycholesterol showed increased ROS compared to control cells.

In addition, referring to FIGS. 4C and 4D, it was confirmed that myeloid leukemia cells (HL60, KG1a, K562 cells) treated with 27-hydroxycholesterol showed increased differentiation (CD11b⁺) compared to control cells.

In addition, referring to FIG. 5A, it can be seen that 27-hydroxycholesterol increases the ER stress response pathway in THP1 cells.

Through this, it was confirmed that the 27-hydroxycholesterol increased the apoptosis and ROS in hematopoietic stem and progenitor cells. Specifically, referring to an apoptosis inducing mechanism of 27-hydroxycholesterol in FIG. 5B, it can be seen that 27-hydroxycholesterol increases the ROS response and induces the apoptosis through the ER stress response pathway.

### Example 4. Confirmation of Gene Expression Level According to Treatment of 27-Hydroxycholesterol

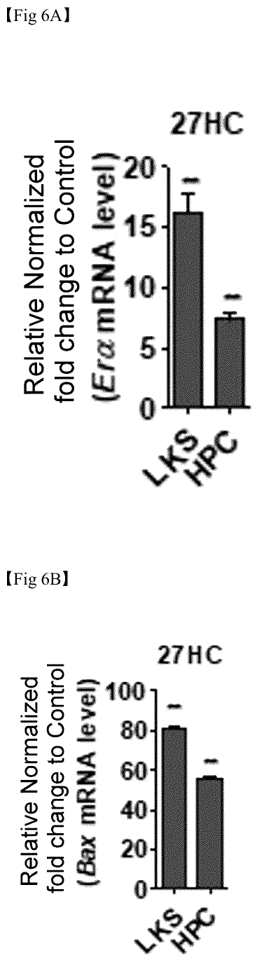

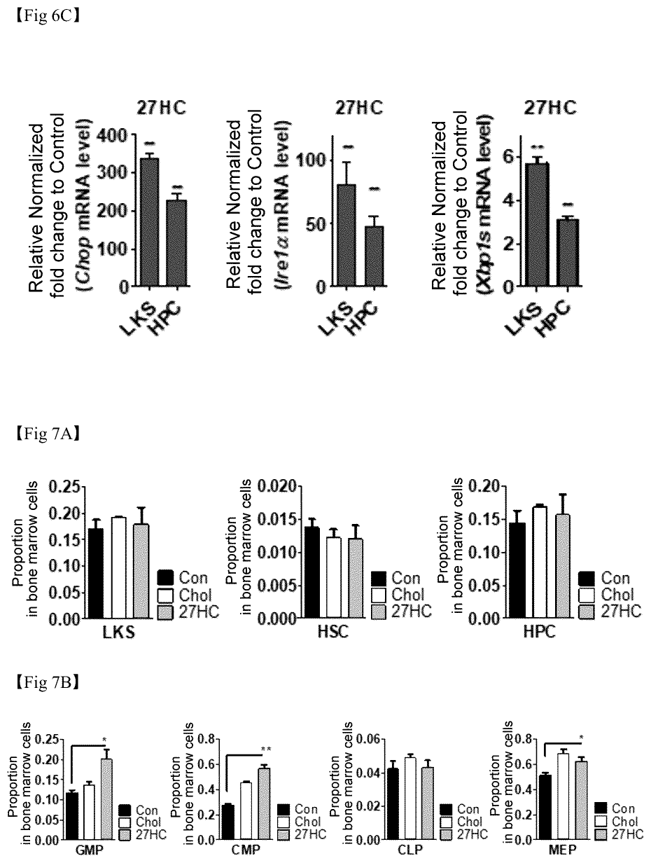

Bone marrow cells were extracted from the mouse tibia and femur and treated with 6.2 μM of 27-hydroxycholesterol for 48 hours. After treatment, the cells were added with PE-Cy5-CD3, PE-Cy5-CD4, PE-Cy5-CD8, PE-Cy5-CD19, PE-Cy5-B220, PE-Cy5-GR1, PE-Cy5-Ter119, PE-Sca1, APC-Add cKit, PE-Cy7-CD150, and APC-Cy7-CD48 antibodies, stained for 30 minutes at 4° C., and then LKS (Lin-Sca1⁺cKit⁺) and hematopoietic progenitor cells (Lin⁻Sca1⁺cKit⁺CD48⁺) were separated using FACSAria. Thereafter, RNA was extracted from the cells using an RNA extraction kit, and cDNA was synthesized from RNA using a cDNA synthesis kit. The synthesized cDNA was subjected to qRT-PCR using primers related to apoptosis and ER stress and SYBR-green. As a result, it was confirmed that gene expression related to apoptosis and ER stress was increased according to treatment of 27-hydroxycholesterol in LKS and hematopoietic progenitor cells, which were immature cells, compared to total bone marrow cells, as illustrated in FIG. 6.

Referring to FIG. 6A, it can be seen that the expression of an Era gene related to cancer cell growth in LKS cells and hematopoietic progenitor cells (HPC) was increased by treating 27-hydroxycholesterol.

In addition, referring to FIG. 6B, it can be seen that the expression of a Bax gene related to apoptosis in LKS cells and hematopoietic progenitor cells (HPC) was increased by treating 27-hydroxycholesterol.

In addition, referring to FIG. 6C, it can be seen that the expression of Chop, Ire1α, and Xbp1s genes related to ER stress in LKS cells and hematopoietic progenitor cells (HPC) was increased by treating 27-hydroxycholesterol.

### Example 5. In Vivo Cytotoxicity Test of 27-Hydroxycholesterol

In order to confirm whether 27-hydroxycholesterol had cytotoxicity in vivo, ethanol, cholesterol, and 27-hydroxycholesterol were injected into the abdominal cavity of the mouse at a concentration of 20 mg/kg, and after 24 hours, bone marrow cells were extracted from the mouse tibia and femur using an FACS staining buffer and attached with antibodies, and then the cells were analyzed with FACSCanto2 equipment. As a result, the 27-hydroxycholesterol showed no significant change in vivo. Through this, it was confirmed that the 27-hydroxycholesterol does not exhibit cytotoxicity in vivo, as illustrated in FIG. 7.

### Example 6. Comparison of Leukemia Cell Growth Between 27-Hydroxycholesterol and 7α-Hydroxycholesterol in Cells

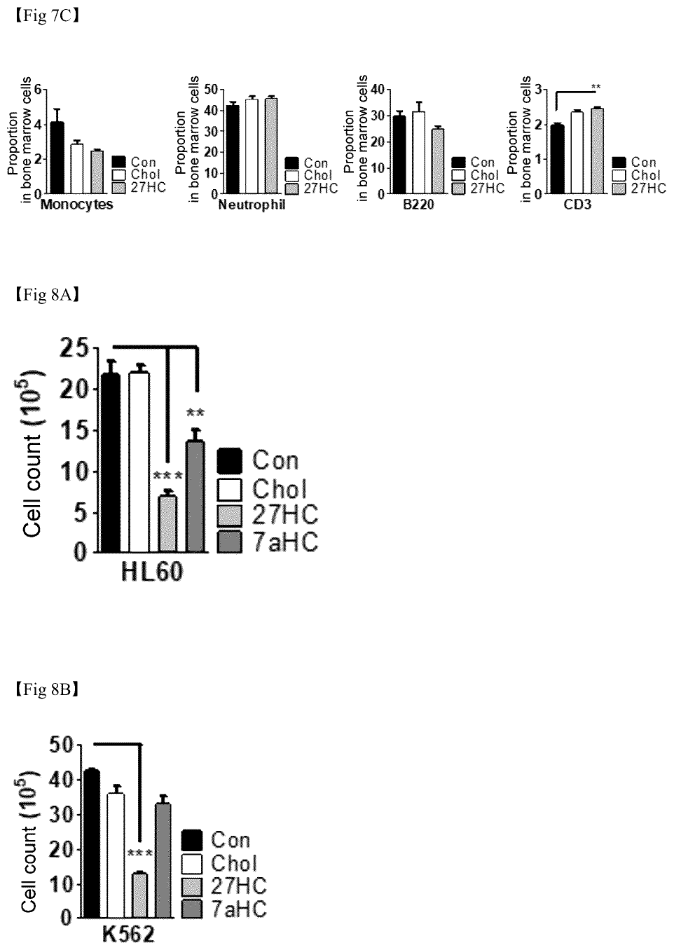

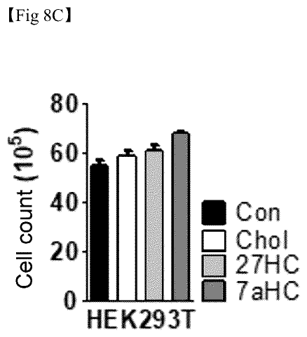

An acute myeloid leukemia cell line HL60, and a chronic myeloid leukemia cell line, K562 cells were treated with 6.2 μM of cholesterol, 7α-hydroxycholesterol, and 27-hydroxycholesterol for 48 hours and then strained with trypan blue, and then the cell count was confirmed. As a result, it was confirmed that 7α-hydroxycholesterol did not have a significant effect on the number of leukemia cell lines, and 27-hydroxycholesterol decreased the number of cells in the leukemia cell lines, as illustrated in FIG. 8 [Con: untreated leukemia cell line, Chol: leukemia cell line treated with cholesterol, 27HC: leukemia cell line treated with 27-hydroxycholesterol, and 7aHC: leukemia cell line treated with 7α-hydroxycholesterol].

Referring to FIGS. 8A and 8B, it was confirmed that in the group treated with 27-hydroxycholesterol (27HC), the growth of leukemia cells (HL60 cells and K562 cells) was inhibited, whereas in the group treated with 27-hydroxycholesterol (27HC), the effect of inhibiting the growth of leukemia cells (HL60 cells and K562 cells) was minimal.

In addition, referring to FIG. 8C, it was confirmed that although being the same oxidized cholesterol, 7α-hydroxycholesterol had no effect on chronic myeloid leukemia cells, and had a weaker effect on acute myeloid leukemia cell lines than 27-hydroxycholesterol, so that 27-hydroxycholesterol suppressed the growth of leukemia cells.

### Example 7. Confirmation of Effect of 27-Hydroxycholesterol on Regeneration and Proliferation of Hematopoietic Stem Cells Ex Vivo

Bone marrow cells were taken out ex vivo, treated with 6.2 μM of 27-hydroxycholesterol (27-HC), and then subjected to bone marrow transplantation.

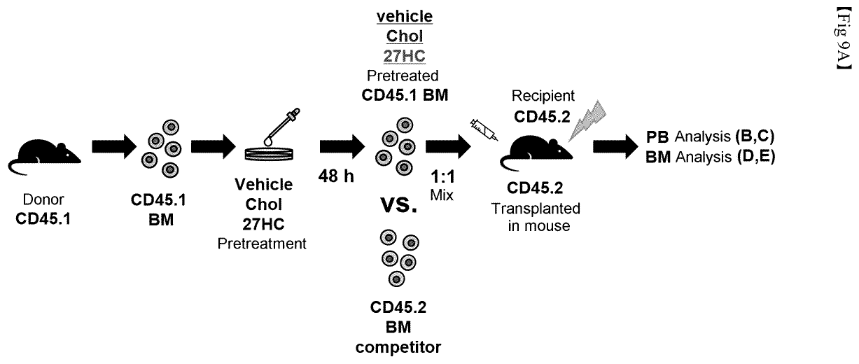

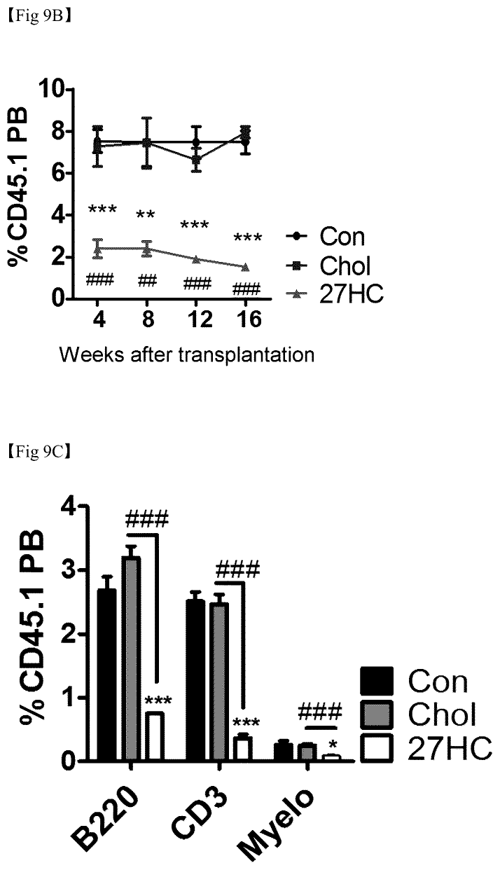

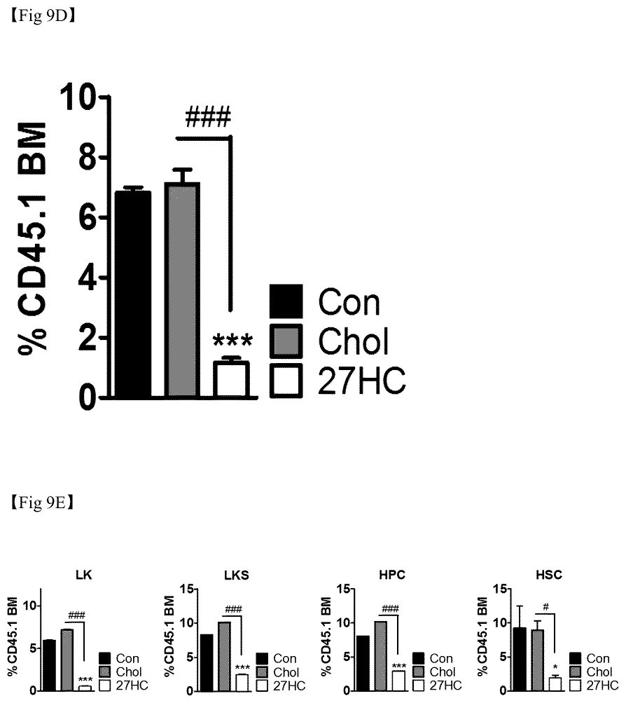

Specifically, referring to FIG. 9A, BM cells were extracted from the tibia and femur of a donor CD45.1 mouse, and then treated with vehicle, 6.2 μM cholesterol, and 6.2 μM 27-hydroxycholesterol for 48 hours. Then, bone marrow transplantation was performed by mixing the bone marrow cells of the CD45.2 mouse in a 1:1 ratio and injecting the mixture into the caudal vein of a recipient CD45.2 mouse that had been irradiated with 4 Gy. One month later, blood was extracted from the veins of the recipient mouse that had received bone marrow transplantation, and the cell proportion of the donor mouse was analyzed. Blood was extracted from the mouse jugular vein and added in 10 mM EDTA to prevent blood coagulation, and added in an ACK lysis buffer to lyse red blood cells. It was confirmed that after attaching CD45.1-APC and CD45.2-FITC antibodies, donor mouse CD45.1 cells were analyzed using FACSCanto2 equipment, and 27-hydroxycholesterol suppressed the regeneration and proliferation of hematopoietic stem cells, as illustrated FIGS. 9A to 9E.

Referring to FIG. 9B, as a result of analyzing the donor mouse CD45.1 cells in blood, it can be seen that the engraftment ability of cells treated with 27-hydroxycholesterol is decreased.

In addition, referring to FIG. 9C, as a result of measuring the proportions of monocytes (CD11b⁺), B cells (B220+), and T cells (CD3+) of a donor mouse in the blood of a recipient mouse after transplantation, it can be seen that the proportions of cells treated with 27-hydroxycholesterol are decreased.

Referring to FIG. 9D, as a result of analyzing the donor mouse CD45.1 cells in bone marrow cells, it can be seen that the engraftment ability of cells treated with 27-hydroxycholesterol is decreased.

In addition, referring to FIG. 9E, as a result of measuring the proportions of LK cells, LKS cells, hematopoietic progenitor cells (HPC), and hematopoietic stem cells (HSC) of the donor mouse in the bone marrow cells of the recipient mouse after transplantation, it can be seen that the proportions of cells treated with 27-hydroxycholesterol are decreased.

Through this, it was confirmed that 27-hydroxycholesterol suppressed the regeneration and proliferation of hematopoietic stem cells in blood and bone marrow cells.

### Example 8. RNA-Sequence Analysis of Acute Myeloid Leukemia Data Sheet GSE116256

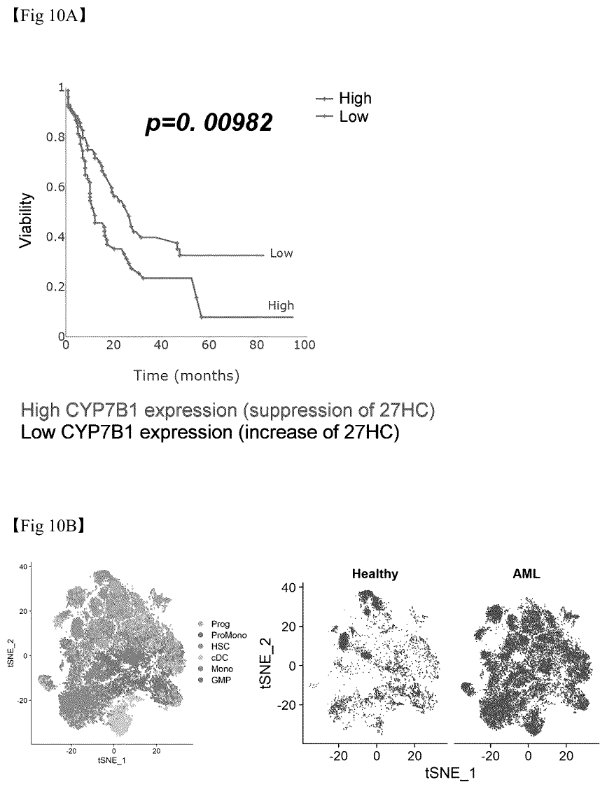

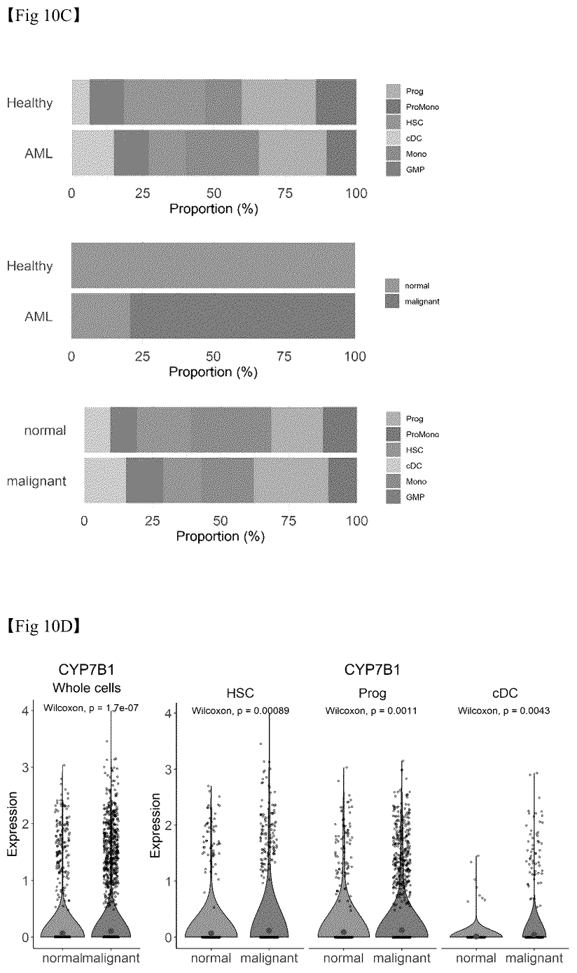

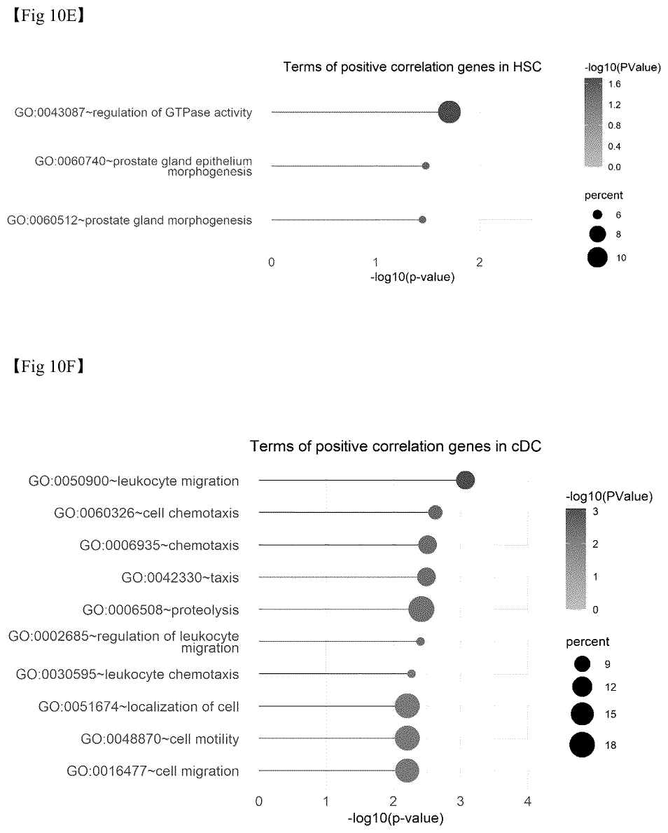

Single cell RNA sequencing was performed using bone marrow cells from a healthy donor and a patient with acute myeloid leukemia to confirm cellular compositions and genetic changes for differential expression of CYP7B1, a metabolic enzyme of 27-hydroxycholesterol, and the results are illustrated in FIG. 10.

Referring to FIG. 10A, as a result of analyzing the viability according to the expression of CYP7B1, a metabolic enzyme that consumed 27-hydroxycholesterol based on the GSE12417 data sheet, the higher the CYP7B1 level, the lower the viability.

In addition, referring to FIG. 10B, the GSE116256 datasheet was analyzed to characterize the expression of CYP7B1 in bone marrow cells from an acute myeloid leukemia patient (AML) and a healthy donor (Healthy), and the cells were classified into 6 cell types.

In addition, referring to FIG. 10C, as a result of analyzing the hematopoietic cell compositions of a patient with acute myeloid leukemia (AML) and a healthy donor (Healthy), referring to an upper graph, it can be seen that the patient with acute myeloid leukemia (AML) has high proportions of dendritic cells (cDC) and monocytes (Mono) and a low proportion of hematopoietic stem cells (HSC). In addition, referring to a middle graph, it can be seen that the patient with acute myeloid leukemia (AML) has a high proportion of malignant cells, and referring to a lower graph, it can be seen that in a cell type proportion of normal and malignant cells in the patient with acute myeloid leukemia (AML), the proportions of dendritic cells (cDC) and progenitor cells (Prog) are high in malignant cells.

In addition, referring to FIG. 10D, it can be seen that the expression of CYP7B1 is high in all malignant cells, hematopoietic stem cells (HSC), progenitor cells (Prog), and dendritic cells (cDC) in the patient with acute myeloid leukemia.

In addition, referring to FIGS. 10E and 10F, it can be seen that when listing the biological processes of genes differentially correlated with CYP7B1 between malignant cells and normal cells, it can be confirmed that the differential expression of CYP7B1 is associated with genes related to GTPase activity and leukocyte migration.

Through this, it is shown that when inhibition of CYP7B1 expression in the patient with acute myeloid leukemia is used together with chemotherapy or radiation therapy for leukemia treatment, a therapeutic effect is better.

Therefore, according to the present invention, the pharmaceutical composition including 27-hydroxycholesterol may suppress the proliferation of hematopoietic stem cells by increasing the active oxygen of the hematopoietic stem cells and activating a cascade of endoplasmic reticulum stress, and promote the apoptosis of blood cancer cells by activating RE1a, eIF2a, and CHOP signaling pathways which were important for the apoptosis of blood cancer cells. As a result, it was confirmed that the pharmaceutical composition may prevent, improve, or treat myeloid leukemia.

The foregoing detailed description illustrates the present invention. Further, the aforementioned contents show and describe the preferred exemplary embodiment of the present invention, and the present invention can be used in various other combinations, modifications, and environments. That is, the foregoing content may be modified or corrected within the scope of the concept of the invention disclosed in the present specification, the scope equivalent to that of the disclosure, and/or the scope of the skill or knowledge in the art. The foregoing exemplary embodiment describes the best state for implementing the technical spirit of the present invention, and various changes required in specific application fields and uses of the present invention are possible. Accordingly, the detailed description of the invention above is not intended to limit the invention to the disclosed exemplary embodiment. Accordingly, the detailed description of the invention above is not intended to limit the invention to the disclosed exemplary embodiment.

1-11. (canceled)

12. A pharmaceutical composition for preventing or treating myeloid leukemia (ML) comprising 27-hydroxycholesterol or a pharmaceutically acceptable salt thereof as an active ingredient.

13. The pharmaceutical composition of claim 12, wherein the 27-hydroxycholesterol suppresses the proliferation of hematopoietic stem cells.

14. The pharmaceutical composition of claim 13, wherein the proliferation of the hematopoietic stem cells is suppressed by endoplasmic reticulum (ER) stress caused by the production of reactive oxygen species (ROS).

15. The pharmaceutical composition of claim 12, wherein the 27-hydroxycholesterol induces the apoptosis of blood cancer cells.

16. The pharmaceutical composition of claim 15, wherein the apoptosis of the blood cancer cells is caused by ER stress induced by increased expression of one or more genes selected from the group consisting of inositolrequiring kinase 1α (IRE1α), eukaryotic translation initiation factor 2α (Elf2α), and C/EBP homologous protein (CHOP).

17. The pharmaceutical composition of claim 12, wherein the concentration of the 27-hydroxycholesterol or the pharmaceutically acceptable salt thereof is 1 to 20 μM.

18. A health functional food composition for preventing or improving myeloid leukemia (ML) comprising 27-hydroxycholesterol or a pharmaceutically acceptable salt thereof as an active ingredient.

19. A method for preventing or treating myeloid leukemia (ML) comprising administering 27-hydroxycholerol to a subject in need thereof.
